(19) United States
(12) Patent Application Publication
(10) Pub. No.: US 2007/0190131 A1
(43) Pub. Date: Aug. 16, 2007

(54) PRESS-FIT RAPID RELEASE MEDICAMENT AND METHOD AND APPARATUS OF MANUFACTURING

(76) Inventor: Ronald L. Perry, Paw Paw, MI (US)

Correspondence Address: PRICE HENEVELD COOPER DEWITT & LITTON, LLP, 695 KENMOOR, S.E., P O BOX 2567, GRAND RAPIDS, MI, 49501, US

(21) Appl. No.: 11/672,584

(22) Filed: Feb. 8, 2007

Related U.S. Application Data
(60) Provisional application No. 60/772,352, filed on Feb. 10, 2006.

(51) Int. Cl. A61K 9/48

(52) U.S. Cl. 424/452; 249/187.1; 425/177

(57) ABSTRACT

A caplet having press-fit gelatin capsule shell halves includes one or more dimples formed in one or both ends of the caplet shells, which significantly reduces the thickness of the gelatin which thereby dissolves more quickly, allowing the medicament to be rapidly released into the body's digestive system. In one embodiment of the invention, the closing pins used to press the conventional capsule shell halves onto a core include a raised projection for debossing the dimples. As holding blocks of a press-fit machine move to encapsulate a caplet, closing pins form compressed dimples in at least one or preferably both ends of the gelatin capsule shell as the shells are applied to the core.

### CROSS-REFERENCE TO RELATED APPLICATIONS

This application claims priority under 35 U.S.C. § 119(e) on U.S. Provisional Application No. 60/772,352 entitled PRESS-FIT RAPID RELEASE MEDICAMENT AND METHOD OF MANUFACTURE, filed on Feb. 10, 2006, by Ronald L. Perry, the entire disclosure of which is incorporated herein by reference.

### BACKGROUND OF THE INVENTION

The present invention relates to a coated medicament which has one or more indentations formed in the coating to allow the coating to rapidly dissolve to release medicament contained therein and a method and apparatus for manufacturing such a medicament.

Typically, medicaments, such as analgesics including, for example, aspirin, acetaminophen, ibuprofen, NSAIDS or the like, are sold in a variety of dosage forms. The medicament itself is typically formed in the shape of a compressed circular tablet or a caplet-shaped tablet which frequently is coated with a hypromellose and hydroxypropyl cellulose (HPC) coating, such as Opadry®. Consumer studies have shown that consumers prefer a gelatin coating for such medications to provide easier swallowing and a better mouth feel as compared to uncoated medicaments, even though the uncoated (except for an Opadry® coating almost universally employed) medicament provides a faster, more rapid release of the medication when swallowed.

In order to accommodate the consumer desire for a gelatin-coated product, numerous techniques have been employed for gelatin coating medicament tablets. Such techniques include pan coating, dip coating, enrobing, and spray coating of gelatin onto a core, which can be circular conventional tablet shape, a caplet tablet shape, or any other desired shape for a swallowable medicament. When caplets are covered with gelatin shells, they can be press-fit or shrunk fit onto a core as, for example, shown by U.S. Pat. Nos. 5,415,868 and 5,824,338.

A partially dip-coated product, such as disclosed in FIG. 1B of U.S. Pat. No. 5,234,099, provides gelatin coating on opposite ends of, for example, a caplet, but leaves a center band of the core exposed, thereby having the benefit and mouth feel of a gelatin-coated product while having a faster or more rapid release characteristic of an uncoated, less consumer-desirable dosage form.

Another approach to the partial dip-coating of a caplet-shaped tablet is partial encapsulation by press-fitting shortened capsule halves onto the core of a caplet. Such construction is disclosed in pending PCT patent application entitled QUICK DISSOLVE CAPSULE AND METHOD OF MANUFACTURING, Application No. PCT/US2005/031962, filed on Sep. 8, 2005, and assigned to the Assignee of the present invention. Although this dosage form has the same benefits as the partially dip-coated medicament, namely, the ease of swallowing and preferable mouth feel, it requires the use of specially manufactured capsule shell halves, which are somewhat shorter than existing capsule shell halves employed in press-fit caplet manufacturing machines. The machines may also have to be modified to accommodate certain capsule shell halves.

Thus, there remains a need for a unique dosage form which has the benefits of a press-fit gelatin-coated medicament and yet has the rapid release characteristics approaching that of an uncoated caplet.

### SUMMARY OF THE INVENTION

The gelatin covered core, method of manufacturing, and apparatus for manufacturing a gelatin covered core of the present invention satisfies this need by providing a caplet-shaped core having press-fit gelatin capsule shell halves which abut at their free ends when press-fit onto the core to completely encapsulate the core. The shell halves include one or more dimples formed in one or more ends of the caplet shells to significantly reduce the thickness of the gelatin. As a result, the gelatin in the dimpled area(s) dissolves more quickly, allowing the core material to be rapidly dissolved. When used for a medicament, the active ingredients are rapidly released into the body's digestive system upon swallowing. In one embodiment of the invention, the closing pins used to press-fit the conventional capsule gelatin shell halves onto a core include a raised projection. When the closing pins move to encapsulate a caplet, they form compressed dimples in at least one or preferably both ends of the gelatin capsule shell as the shells are applied to the core. The dimples have a significantly reduced gelatin thickness, which dissolves more quickly, allowing rapid release of the medicament in the area of the dimples.

The press-fit equipment is modified, although the sequence of operation is substantially the same as existing press-fit sequences of operation. The press-fit machinery includes upper and lower closing pins which force-fit capsule shells onto a core held in a block with at least one of the upper and lower closing pins including a projection extending in a direction toward the medicament core along the longitudinal axis thereof. When the upper and lower pins compress the gelatin capsule shells onto the core, at least one dimple is formed in one end of the medicament. This significantly reduces the thickness of the gelatin at the location of the projection in the closing pin and ultimately in the completed caplet. In a preferred embodiment of the invention, both the upper and lower closing pins of the press-fit machine include raised projections for forming dimples on opposite ends of the gelatin shells press-fit onto the caplet core. In other embodiments, a plurality of dimples may be formed in each of the closing pins.

Thus, the invention contemplates the provision of a press-fit gelatin covered medicament having a gelatin coating with at least one dimple formed in the coating to greatly reduce the thickness of the gelatin. In a preferred embodiment, the medicament is in the form of a caplet-shaped core having press-fit gelatin capsule shells forced thereon utilizing closure pins of a press-fit machine, wherein at least one of the pins includes a projection extending toward the core as the capsule shell is press-fit onto the core. The invention also contemplates the resultant medicament as well as specialized closure pins which include projections on a face of the pin engaging a gelatin capsule shell half and the method of manufacturing a medicament by forcing gelatin capsule shell halves over a caplet-shaped core while simultaneously forming dimples in an end of at least one of the capsule shells.

These and other features, objects and advantages of the present invention will become apparent upon reading the following description thereof together with reference to the accompanying drawings.

### BRIEF DESCRIPTION OF THE DRAWINGS

FIG. 1 is a vertical cross-sectional view of a medicament embodying one embodiment of the present invention;

FIG. 2 is a right end view of the medicament shown in FIG. 1, it being understood that the left end view is substantially the same;

FIG. 3 is a greatly enlarged fragmentary view, taken in the encircled area III of FIG. 1;

FIG. 4 is a vertical cross-sectional view of one of the press-fit stations of a machine, showing the configuration of the closure pins employed in the manufacture of the medicament shown in FIGS. 1-3; and

FIG. 5 is an end view of one end of an alternative embodiment of the invention.

### DETAILED DESCRIPTION OF THE PREFERRED EMBODIMENT

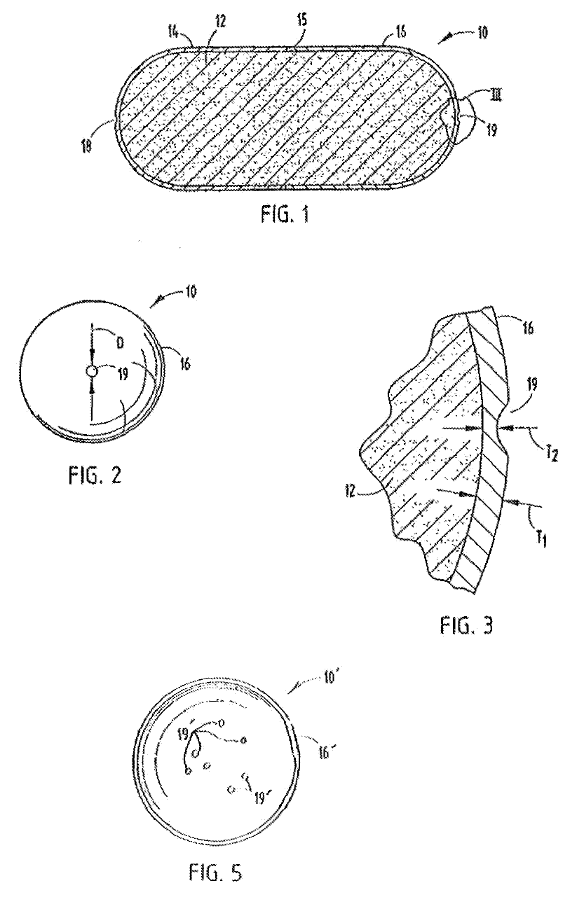

Referring initially to FIG. 1, there is shown a medicament 10 embodying the present invention and comprising a tablet having a caplet-shaped core 12 which is compressed by conventional equipment to form a core with suitable excipients and active ingredients. Core 12 may be any number of medicaments, such as analgesics including aspirin, ibuprofen, acetaminophen, and NSAIDS or any number of other medicaments, such as anesthetics, antiarthritics, antibiotics, anticoagulants, antidepressants, antidiabetic agents, antiemetics, antiflatulents, antifungals, antihistamines, anti-infective agents, anti-inflammatory agents, antispasmodics, antitussives, antivirals, appetite suppressants, bronchodilators, cardiovascular agents, central nervous system agents, central nervous system stimulants, decongestants, diuretics, expectorants, gastrointestinal agents, migraine preparations, motion sickness products, mucolytics, muscle relaxants, oral contraceptives, osteoporosis preparations, polydimethylsiloxanes, respiratory agents, sleep-aids, urinary tract agents, and mixtures of the above as active ingredients mixed with conventional excipients, including fillers, disintegrating agents, lubricants, sweetening agents and/or flavorants. The following are examples of preferred embodiments of medicament cores using either a super disintegrant or effervescent couple to assure the rapid release of the medicament when manufactured according to the teaching of this invention.

| | | |
|---|---|---|
| Ingredients | mg/tab | % |

EXAMPLE 1:

| | | |
|---|---|---|
| APAP Compap Course L | 555.500 | 88.174603 |
| (90% acetaminophen) | | |
| Copovidone S-630 | 11.500 | 1.825397 |
| Crospovidone XL | 63.000 | 10.000000 |

EXAMPLE 2:

| | | |
|---|---|---|
| APAP Compap Course L | 555.500 | 87.757 |
| (90% acetaminophen) | | |
| Microcryst Cellulose | 14.500 | 2.291 |
| Crospovidone XL | 63.000 | 9.953 |

EXAMPLE 3:

| | | |
|---|---|---|
| APAP Compap Course L | 555.500 | 81.811 |
| (90% acetaminophen) | | |
| Microcryst Cellulose | 14.500 | 2.135 |
| Sod Bicarb #2 F-gran | 54.500 | 8.027 |
| Citric Acid Anhydrs | 54.500 | 8.027 |

EXAMPLE 4:

| | | |
|---|---|---|
| APAP Compap Course L | 555.500 | 77.910 |
| (90% acetaminophen) | | |
| Microcryst Cellulose | 14.500 | 2.034 |
| Crospovidone XL | 34.000 | 4.769 |
| Sod Bicarb #2 F-gran | 54.500 | 7.644 |
| Citric Acid Anhydrs | 54.500 | 7.644 |

The medicament 10 of the preferred embodiment of the invention includes a press-fit gelatin capsule shell 14 on one end and a press-fit gelatin capsule shell 16 at the opposite end, which shells 14 and 16 meet and are abuttingly joined tightly together along seam 15 so as to completely encapsulate the core 12 within the gelatin shells 14 and 16. The gelatin shells initially have a moisture content of from about 15.5% to about 17% to allow the plasticity for the shells to be coupled to core 12. During the press-fitting of the shells onto core 12 at the ends of each of the shells 14 and 16, there is formed a dimple 18 in shell 14 and 19 in shell 16 which is formed by the machine and process described below. As used herein, dimple means a concave indentation or depression formed in the gelatin or other shell material which substantially reduces the cross-sectional thickness of the shell material. Substantially, reduction, as used herein, means from about 50% to about 0% of the original capsule shell thickness.

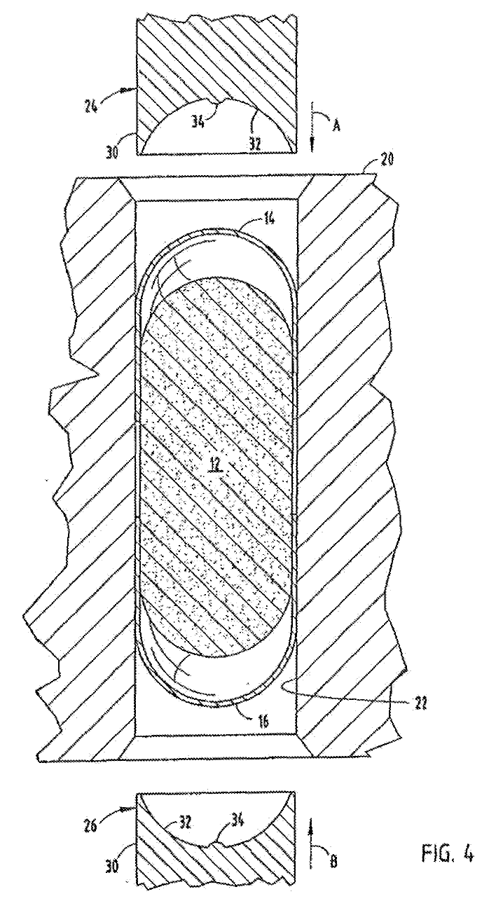

As seen in FIG. 3 in which dimple 19 is shown in greater detail, the thickness of gelatin shell 16 has a thickness T1 of from about 0.020 inches to about 0.060 inches, while the thickness of the dimple formed in the gelatin shell is substantially thinner, shown by the dimension T2 in FIG. 3, with a depth of the circular dimple 19 shown therein formed to a thickness T2 of from about 0.001 mm to about 1 mm. The diameter D (FIG. 2) of the circular dimple 19 can range from about 0.03 mm to about 0.15 mm for a typical 5 mm diameter capsule shell 16. The dimensions of dimple 18 in the opposite end of one embodiment 10 of the medicament is substantially the same as dimple 19 shown in FIGS. 2 and 3. In the broadest form of the invention, the core 12, which typically is a medicament 10, may include only one dimple formed in one end of one of the capsule shell halves 14 or 16, although it is preferable to provide, as shown in FIG. 1, dimples at each end of the medicament 10. The dimples are formed by debossing the gelatin shells 14 and 16 during the press-fit manufacturing of the medicament, as illustrated in FIG. 4.

In FIG. 4, there is shown the holding block 20 of a press-fit encapsulating machine which is commercially available and may be a machine such as a Zanasi 70C manufactured by Industria Macchine Automatiche (IMA). Block 20 represents one of several blocks which are rotated on a rotary turntable, with each block being slightly arcuately shaped and including, for example, eleven apertures, such as aperture 22 (FIG. 4), for holding capsule shell halves, such as 14 and 16, and core 12 therein in position for press-fitting the shell halves 14 and 16 over core 12 utilizing upper and lower closing pins 24 and 26. Pins 24 and 26 move in a direction toward one another, as shown by arrows A and B in FIG. 4, with cylindrical aperture 22 in a direction along the longitudinal axis of the elongated capsule-shaped core 12.

The closure pins 24, 26 each include a generally cylindrical body 30 with a hemispherical depression 32 at the end which extends within aperture 22 and which engages the gelatin shells 14 and 16. Formed in the bottom of the concave hemispherical end 32 in the embodiment shown is a convex generally hemispherical projection 34 in each of the closure pins 24 and 26. During the press-fit operation, projections 34 engage the gelatin shells 14 and 16 forming dimples 18 and 19, respectively, due to the opposing pressure of the closure pins against the shells and caplet core 12. The pressure encountered is the standard pressure employed in the commercially available press-fit machine, and, as an example, the length of the combined strokes of the closure pins (depending on capsule size) ranges from about 0.756 inches to about 0.804 inches for a size 500 capsule. The closure pins, when fully engaged with the core, press-fit the capsule shell halves together to form the medicament shown in FIG. 1 and plastically deform the gelatin shells at the ends to form dimples 18 and 19, respectively, having a relatively thin cross section as compared to the remainder of the walls of shells 14 and 16. When the medicament is swallowed, the reduced cross section thickness of the dimpled areas dissolve more quickly, allowing the gastric juices to dissolve the medicament contained in core 12 more quickly than a conventional press-fit gelatin caplet. Through tests, it has been discovered that the disintegration rate is generally less than about 90 seconds, which is significantly less than that of a conventional press-fit gelatin-shell covered caplet.

If desired, a greater number of dimples 34 can be formed in the upper and lower closure pins 24 and 26 and anywhere from about 1 to about 5 is contemplated. Although the shape is preferably generally convex hemispherical, as shown by projection 34, it is possible to provide other geometric configurations for the dimple-forming projections, including a generally pyramidal projection which would substantially reduce the dimension T2 to approaching zero. FIG. 5 shows a caplet-shaped medicament 10′ with a gelatin shell 16′ having such a plurality of dimples 19′ formed therein at an end of increase the dissolution rate. Each end of the medicament 10′ may include a greater or fewer number of such indentations, which can be of the same size as discussed above.

The press-fit encapsulating machine 100 and its operation are well known, however, a brief description follows. The machine includes a turntable which rotates a plurality of somewhat arcuate shaped capsule holding blocks 20 (FIG. 4) through multiple processing stations by the rotation of the turntable. The machine includes a lower cap inserting station which inserts a capsule shell half 16 (FIG. 4) in the lower portion of each aperture 22 in each of the holding blocks 20. Next, at tablet-loading stations, a caplet 12 is positioned into the upper end of each aperture 22 such that it aligns and at least partially extends within the open end of the lower capsule shell half 16, as illustrated in FIG. 4. The machine also includes an upper shell half loading station, adjacent the tablet station, which inserts capsule shell half 14 into the open end of each of the apertures 22 in block 20 generally in alignment with and extending partially over the caplet 12. The machine also includes a checking station which checks to determine that both capsule shell halves and caplets have been inserted into the apertures 22 in holding block 20. Finally, the machine includes a closing station in which a plurality of upper closing pins 24 align with the apertures 22 in holding block 20 are brought downwardly into aperture 22 while aligned lower closing pins 26 are brought upwardly into the block 20, as best illustrated in FIG. 10. As the turntable rotates, each of the stations operate sequentially to perform their particular task during encapsulation. The upper and lower closing pins are substantially the same as the conventional pins of the commercially available machine with the exception of the modification of the hemispherical ends of the upper and lower pins, as described above, to include centered projections 34 which form the dimples 18 and 19 at the ends of the capsule shell halves.

It will become apparent to those skilled in the art that these and various modifications to the preferred embodiment and method of manufacturing the medicament of the invention as described herein can be made without departing from the spirit or scope of the invention as defined by the appended claims.

The invention claimed is:

1. A gelatin covered core comprising:
a dissolvable core; and
a pair of gelatin shells press-fitted onto said core, wherein at least one of said shells includes an indentation formed therein to reduce the thickness of said gelatin in the area of said indentation to allow rapid release of the contents of said dissolvable core.

2. The gelatin covered core as defined in claim 1 wherein said indentation is at an end of said gelatin shell.

3. The gelatin covered core as defined in claim 2 wherein said indentation is generally circular having a diameter of from about 0.03 mm to about 0.15 mm.

4. The gelatin covered core as defined in claim 3 wherein said gelatin shell has a thickness of from about 0.020 inches to about 0.060 inches and the thickness of the shell at said dimple is from about 0.001 mm to about 1 mm.

5. The gelatin covered core as defined in claim 1 wherein said core is a medicament.

6. The gelatin covered core as defined in claim 1 wherein said core is caplet shaped.

7. The gelatin covered core as defined in claim 1 wherein said core is a caplet-shaped medicament and includes a plurality of indentations in at least one of said shells.

8. The gelatin covered core as defined in claim 1 wherein said indentation has a depth of from about 50% to about 100% of the original gelatin shell thickness.

9. The gelatin covered core as defined in claim 1 wherein each of said shells include at least one indentation at an end thereof.

10. A closure pin design for a press-fit caplet encapsulating machine comprising:
a cylindrical body having an end for engaging a capsule shell, said end including a concave surface and further including a convex projection extending from said concave surface to engage a capsule for forming a dimple therein.

11. The closure pin design as defined in claim 10 wherein said concave surface is generally hemispherical.

12. The closure pin design as defined in claim 11 wherein said projection is centered in said concave surface.

13. The closure pin design as defined in claim 12 wherein said convex projection is generally hemispherical.

14. The closure pin design as defined in claim 10 and further including a plurality of projections.

15. An apparatus for assembling gelatin shell halves onto a caplet-shaped core comprising:
at least one processing station for sequentially inserting a gelatin shell half in a holding block, a caplet-shaped core in said holding block, and a second gelatin shell half onto said caplet-shaped core; and
first and second closure pins movable in opposite directions in alignment with said holding block for compressing said gelatin shell halves onto said caplet-shaped core wherein each of said closure pins include a cylindrical body having an end for engaging a capsule shell, said end including a concave surface and further including a convex projection extending from said concave surface to engage a capsule shell for forming a dimple therein.

16. The apparatus as defined in claim 15 wherein said concave surface is generally hemispherical.

17. The apparatus as defined in claim 16 wherein said convex projection is generally hemispherical.

18. The apparatus as defined in claim 15 wherein each of said closure pins include a plurality of projections.

19. A method of assembling gelatin shell halves onto a caplet-shaped core comprising the steps of:
inserting a gelatin shell half in a holding block;
inserting a caplet-shaped core into said holding block;
inserting a second gelatin shell half onto said caplet-shaped core on an end opposite said first named gelatin shell half; and
moving first and second closure pins in opposite directions for compressing said gelatin shell halves onto said caplet-shaped core wherein each of said closure pins include a cylindrical body having an end for engaging a capsule shell, said end including a concave surface and further including a convex projection extending from said concave surface to engage an end of said capsule shells for forming a dimple therein.

20. The method as defined in claim 19 wherein the closure pins include a plurality of projections for forming a plurality of dimples during the moving step.
